(19) United States
(12) Patent Application Publication
(10) Pub. No.: US 2007/0292405 A1
(43) Pub. Date: Dec. 20, 2007

(54) Uses of Erss Modulators

(76) Inventors: Janathan Seckl, Edinburgh (GB); Cecile Pascale Martin, Edinburgh (GB)

Correspondence Address: MYERS BIGEL SIBLEY & SAJOVEC, PO BOX 37428, RALEIGH, NC, 27627, US

(21) Appl. No.: 10/589,910

(22) PCT Filed: Feb. 21, 2005
(86) PCT No.: PCT/GB05/00648

§ 371 Date: Aug. 8, 2007

(30) Foreign Application Priority Data
Feb. 21, 2004 (GB) 0403889.9

(51) Int. Cl. A61K 38/44; A61K 31/56; A61P 35/00; C12P 21/04; C12Q 1/68; G01N 33/536; G01N 33/48; C12Q 1/26; C12P 19/34; A61K 38/43

(52) U.S. Cl. 424/94.4; 435/25; 435/6; 435/7.92; 435/71.1; 514/178

(57) ABSTRACT

The present invention relates to uses of ERβ modulators in the preparation of medicaments for preventing and/or treating hormone dependant cancers and other proliferative disorders, as well as diagnosis of hormone dependant cancers and other proliferative disorders. The present invention also teaches a method of screening agents for their use in preventing and/or treating hormone dependant cancers and other proliferative disorders.

### FIELD OF THE INVENTION

The present invention relates to uses of ERβ modulators in the preparation of medicaments for preventing and/or treating hormone dependant cancers and other proliferative disorders, as well as diagnosis of hormone dependant cancers and other proliferative disorders. The present invention also teaches a method of screening agents for their use in preventing and/or treating hormone dependant cancers and other proliferative disorders.

### INTRODUCTION

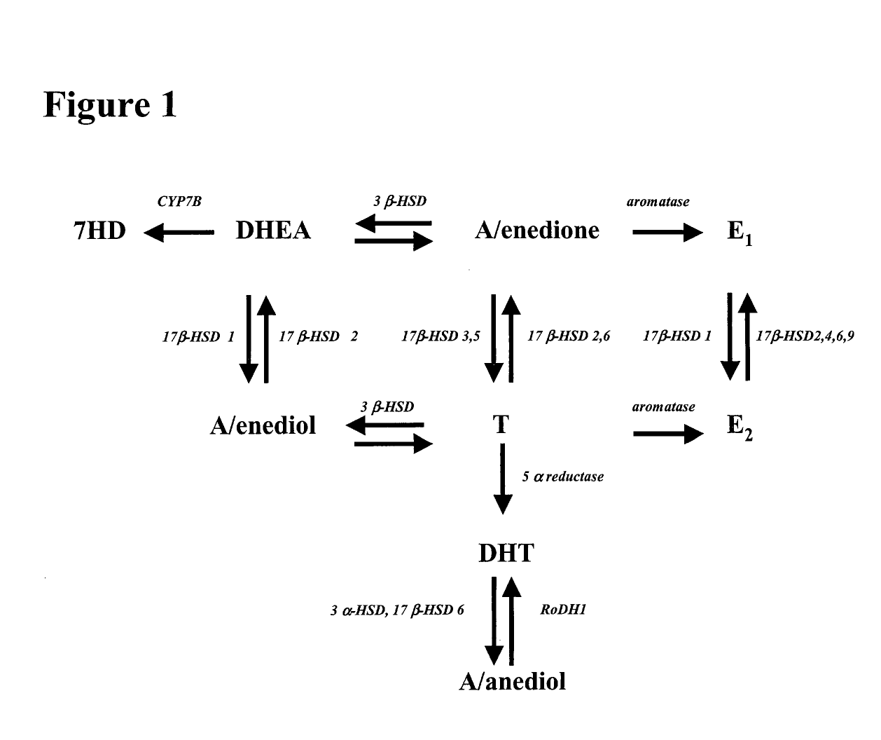

Androgens play an important role in the development and function of many glands, for example the breast, brain and prostate, and are also involved in the initiation and maintenance of hormone dependant cancers and other proliferative disorders for example, prostate cancer (PCa) and benign prostatic hyperplasia (BPH) (1). Recent evidence in animal models and humans have suggested that other steroid hormones including estrogens, may also be involved both in the growth and disorders of glands such as the prostate (2, 3). With age, declining testicular function leads to lower levels of plasma testosterone (T) (4). However, the levels of estrogens (17β-estrodiol: E₂) are maintained by enhanced aromatisation of adrenal androgens, notably dehydroepiandosterone (DHEA) (5) (See FIG. 1), in peripheral tissue such as adipose tissue (6). Therefore in elderly men, the ratio between free E₂ and free T may increase by up to 40% (7). The endocrine changes at mid-life have long been observed and may have an association with the pathologies seen in glands such as the prostate after the 5^{th} decade (1, 8, 9).

In the prostate, estrogens interact with two forms of estrogen receptor (ER), ERα and ERβ. The two receptors differ in their binding affinity for a variety of estrogenic compounds and in their sub-localisation in the human prostate (10-13). ERα is expressed only at low levels and is confined to the stroma, where it may influence epithelial growth in a paracrine manner (14). In contrast, ERβ is highly expressed in prostatic epithelium (13). The role of ERβ in human prostate is not clear. In one study, ERβ knockout mice have been reported to develop prostatic hyperplasia with age (15), suggesting anti-proliferative functions. However, other ERβ knockout models do not have this prostate phenotype. Although these studies show distinct roles for ERα and ERβ, they do not establish the identity of the ligand responsible for these estrogenic actions. Whilst E₂ may be the major ligand for ERβ in most target cells, the levels of E₂ in human prostate are probably too low to activate the receptors (16). This raises the possibility that other estrogenic ligands produced locally in the prostate might be responsible for ERβ estrogenic activity. Indeed, local steroid metabolism is an important determinant of steroid action in various organs (17). Thus, local metabolism in the prostate may be a key to steroid biological activity.

Recently, it has been shown that oxysterol 7α-hydroxylase (CYP7B), a novel cytochrome P450 identified in rodent hippocampus (18-19) and which catalyses the 7α-hydroxylation of DHEA to 7α-hydroxyDHEA (7HD), is highly expressed in rodent prostate (20). CYP7B is the only route for the 7α-hydroxylation of DHEA, as confirmed by the CYP7B knockout animals (21-23), which show no residual DHEA 7α-hydroxylation in prostate and brain (22). CYP7B is unusual amongst P450s in being much more highly expressed in specific extra-hepatic tissues, notably hippocampus and prostate (19) than in the liver. 7α-hydroxylase activity has also been reported in humans (24), but the enzyme(s) responsible for this reaction in most tissues is unknown.

WO97/37664 discloses the use of 7α-substituted steroids and the enzyme CYP7B to treat neuropsychiatric, immune or endocrine disorders, however no mention of the use of such steroids or the enzyme CYP7B for the treatment of prostate disorders is disclosed therein.

The present invention is based upon observations by the present inventors that the expression of CYP7B in human prostate is a major route for DHEA metabolism producing 7αHD.

### SUMMARY OF THE INVENTION

Generally speaking the present invention relates to agents that modulate the activity of ERβ. More specifically the invention relates to the 7-hydroxylated steroids, capable of modulating ERβ, and enzymes that produce 7-hydroxylated steroids. As the level of enzymes capable of catalysing the production of 7-hydroxylated steroids fall (in for example an aged person or person with a prostate disorder), the balance between estrogens and androgens in the prostate may change in favour of the androgenic pathways resulting in a decrease in production of 7-hydroxylated steroids (ERβ agonist). Concomitantly any decrease in expression of an enzyme capable of catalysing the production of a 7-hydroxylated steroid increases the availability of, for example, native DHEA within the prostate for synthesis of potent androgens.

It is postulated that ERβ has the capacity to repress the transcriptional activity of ERα. Moreover by binding to ERβ, it is possible that 7-hydroxylated steroids modulate ERα activity in the stroma compartment and therefore may control the growth of the stroma cells. Also, ERβ may play a role in the differentiation and proliferation of the prostate cells as well as modulating both the initial phases of prostate carcinogenesis and androgen-dependent tumour growth. Thus, 7-hydroxylated steroids and enzymes capable of catalysing the production of 7-hydroxylated steroids may have a significant role in the regulation of the intraprostatic concentration of active steroids and may be a useful tool in the prevention or clinical management of hormone dependant cancers and other proliferative disorders for example prostate disorders.

The 7-hydroxylated steroids are thought to be agonists for the estrogen receptor ERβ. ERβ expression is observed in a number of tissues for example the brain, breast and in particular the epithelium of the prostate (13). Another estrogen receptor with similar distribution in vivo, ERα, is also expressed at low levels in the stroma of the prostate. It has been shown that upon activation, ERβ has the capacity to repress the transcriptional activity of ERα. It is an observation of the present inventors that 7-hydroxylated steroids preferentially bind and modulate ERβ in the prostate epithelium. As such 7-hydroxylated steroids may have the effect of repressing the transcriptional activity of ERα and consequently may control the growth of the stroma cells. It is likely therefore that 7-hydroxylated steroids or other compounds that bind and modulate the activity of ERβ in the epithelium may be useful in the treatment and/or prevention of hormone dependant cancers and other proliferative disorders for example, prostate cancer (PCa) and benign prostatic hyperplasia (BPH).

Thus in a first aspect there is provided use of an ERβ modulator for the preparation of a medicament for the prevention and/or treatment of hormone dependant cancers and other proliferative disorders.

By modulator it is meant any agent that either antagonises or agonises ERβ. Preferably the modulator is an ERβ agonist.

In a further aspect, the present invention provides use of 7-hydroxylated steroids and/or enzymes that produce 7-hydroxylated steroids for the preparation of a medicament for the prevention and/or treatment of hormone dependant cancers and other proliferative disorders.

Preferred steroids useful in the preparation of such a medicament include 7α-hydroxylated and 7β-hydroxylated steroids and more specifically, for example, 7α-hydroxy-DHEA (7DH), 7α-hydroxy-pregnenolone, 7α-hydroxy-β-estradiol, 7α,3β,17β-androstenetriol, 7α,3β,17β-androstanetriol, plus 7α-hydroxycholesterol, 7α-25-hydroxycholesterol, 7α-24-hydroxycholesterol, 7α-27-hydroxycholesterol and other 7α-di-hydroxy and 7α-multi-hydroxylated forms of cholesterol.

Such a treatment may involve administering an amount of either a 7-hydroxylated steroid or an enzyme capable of catalysing the production of a 7-hydroxylated steroid in a subject, in association with a pharmaceutically acceptable carrier or diluent. This may be formulated, for example, in a form suitable for gastrointestinal (e.g. oral), transmucosal, parenteral, transdermal, inhalation or topical administration or administration as a suppository, to a patient in need of such treatment to prevent and/or treat a hormone dependant cancer or other proliferative disorder. Preferably the route of administration should favour the appropriate gland, for example the prostate, as the target for the 7-hydroxylated steroid or the enzyme capable of catalysing the production of a 7-hydroxylated steroid.

It is thought that the effect of administering to a patient either a compound capable of modulating ERβ, a 7-hydroxylated steroid or an enzyme capable of catalysing the production of a 7-hydroxylated steroid, is that of redressing the balance between the estrogens and the androgens in a diseased prostate and thus modulating ERα activity to, for example, control the growth of the stroma cells in the prostate. In the case of, for example, the treatment of a prostate disorder, direct or local administration to the prostate, or in the vicinity of the prostate may be preferred so as to not effect, or minimally effect 7-hydroxylated steroids or enzymes capable of catalysing the production of a 7-hydroxylated steroid formed at other sites of the body, for example in the brain.

Examples of hormone dependant cancers and other proliferative disorders potentially treatable by the abovementioned medicament/formulations include disorders of the prostate, for example Benign Prostatic Hyperplasia (BPH), Prostatitis and Prostate Cancer (PCa), disorders of prostate development and of prostate ageing as well as disorders such as Breast Cancer.

Examples of enzymes that would function in the desired manner include the P450 cytochrome enzyme CYP7B as disclosed in WO97/37664 to which the skilled reader is directed. However it is recognised that a person skilled in the art using well established techniques would be able to manipulate said enzyme in a number of ways such that the activity of the enzyme may be modified. Examples of enzyme modification could include modification of the amino acids at the active site in order to provide greater affinity for the substrate. This could be achieved using techniques well known in the art such as site-directed mutagenesis or other PCR-based procedures (Maniatis et al, 1989). Details of such modification procedures are also given in WO97/37664.

By enzyme it is understood that this will include the protein, peptides, fragments or portions thereof and the nucleic acids encoding said proteins, peptides, fragments or portions thereof. It is understood that the proteins, peptides, fragments or portions thereof are also capable of catalysing the production of a 7-hydroxylated steroid, for example CYP7B.

All proteins, peptides, fragments or portions thereof mentioned herein may be expressed, for example, by recombinant means. That is expressible nucleic acid encoding said proteins, peptides, fragments or portions thereof may be introduced into appropriate cells such as bacterial, for example Escherichia coli, and eukaryotic, for example yeast, insect or mammalian cells. Said proteins may also be purified from cells where appropriate, using suitable techniques known in the art. The skilled man would be able to follow the teachings of WO97/37664 to enable the production of an enzyme capable of catalysing the production of a 7-hydroxylated steroid. Specifically WO97/37664 provides the skilled man with the information facilitating the production of CYP7B an enzyme capable of catalysing the production of a 7-hydroxylated steroid, which, as a result of the observations of the present inventors, is potentially useful in the treatment of hormone dependant cancers and other proliferative disorders.

Steroids for use in the treatment and/or prevention of a hormone dependant cancer or other proliferative disorder are 7-hydroxylated steroids, preferably those which are 7α-hydroxylated specifically, for example, 7α-hydroxy-DHEA (7DH), 7α-hydroxy-pregnenolone, 7α-hydroxy-β-estradiol 7α,3β,17β-androstenetriol, 7α,3β,17β-androstanetriol, all 7β-hydroxylated forms thereof, plus 7α-hydroxycholesterol, 7α-25-hydroxycholesterol, 7α-24-hydroxycholesterol, 7α-27-hydroxycholesterol and other 7α-di-hydroxy and 7α-multi-hydroxylated forms of cholesterol. Such steroids may be produced synthetically or by using, for example, recombinantly produced enzymes capable of catalysing the production of said 7-hydroxylated steroid, for example CYP7B. In order to produce a 7-hydroxylated steroid, a suitable substrate may, for example, be added either directly to said enzyme or to, for example, a cell culture or the like. Said cell culture or the like may comprise cells transformed with a vector containing a gene encoding said enzyme or a protein, peptide, fragment or portion thereof also capable of catalysing the production of a 7-hydroxylated steroid from said substrate.

By substrate it is meant any compound capable of being converted to a 7-hydroxylated steroid by an enzyme capable of catalysing the production of a 7-hydroxylated steroid. For example, pregnenolone, dehydroepiandosterone (DHEA), 3beta-androstanediol, 3β-androstenediol and β-estradiol are all suitable substrates capable of being converted to a 7-hydroxylated steroid by an enzyme capable of catalysing the production of a 7-hydroxylated steroid. In a further aspect of the present invention there is provided a method of treatment and/or prevention in a patient suffering from or predisposed to a hormone dependant cancer or other proliferative disorder comprising administering to a patient in need thereof an effective amount of either a 7-hydroxylated steroid and/or an enzyme capable of catalysing the production of a 7-hydroxylated steroid.

In a further aspect of the present invention there is provided a means of treating a patient with an abnormally functioning gene encoding an enzyme capable of catalysing the production of a 7-hydroxylated steroid. By “abnormally” it is meant a gene that functions in a manner different to a gene expressed in a healthy gland, for example the prostate, for example as a result of a mutation, or the down-regulation of said gene by some means, for example repression. Such a treatment may comprise the administration of a suitable vector containing a normally functioning gene encoding said enzyme to the gland. By “normally functioning” it is meant a gene that functions in the same manner as a gene expressed in a healthy gland. Examples of suitable vectors would include plasmids, liposomes, adenovirus, vaccinia or herpes virus vectors modified to include a gene capable of expressing a functional enzyme capable of catalysing the production of a 7-hydroxylated steroid from a suitable substrate.

Preferably a gene therapy vector for use in the treatment of hormone dependant cancers and other proliferative disorders resulting from, for example, a mutated gene encoding an enzyme capable of catalysing the production of a 7-hydroxylated steroid or a gene encoding said enzyme which has become through some mechanism down-regulated, should be administered such that the favoured target may be the appropriate gland. A vector administered in association with a pharmaceutically acceptable carrier with, for example, formulations being suitable for topical, transmucosal, parenteral, transdermal, gasterointestinal (oral) or inhalation administration. Conveniently administration may be by means of parenteral, topical or transmucosal administration such that the vector is delivered directly to, or proximal to the gland.

In another aspect the present invention provides a method of diagnosing in a patient either a level of a 7-hydroxylated steroid or a level of an enzyme capable of catalysing the production of a 7-hydroxylated steroid or detecting a mutation in a sequence encoding an enzyme capable of catalysing the production of a 7-hydroxylated steroid, wherein the method comprises the steps of:
a) obtaining a sample from a patient;
b) detecting a level of 7-hydroxylated steroid or an enzyme capable of catalysing the production of a 7-hydroxylated steroid or ascertaining the sequence of the nucleic acid encoding said enzyme; and
c) comparing said detected level or the sequence of said nucleic acid with a normal level or sequence.

By patient it is meant either a healthy person, a person suspected of having, predisposed to developing, or suffering from a hormone dependant cancers or other proliferative disorder.

It is understood that a sample may be in the form of a biopsy for example a prostate biopsy, or where appropriate could include blood, urine, or semen samples. Blood, for example, may provide a means for the detection of levels of 7α-hydroxylated steroids or enzymes capable of catalysing the production of a 7-hydroxylated steroid in the body generally at the time the sample is taken. A patient suffering from, for example, a prostate disorder of the type detailed above, who upon having a sample taken and tested by the aforementioned assay, is found to have abnormal levels of either 7-hydroxylated steroids or an enzyme capable of catalysing the production of a 7-hydroxylated steroid in their blood, may be eligible for further tests to determine whether the abnormal levels as detected by said assay are due specifically to a disorder of the prostate. It is envisaged that, in the case of a prostate disorder, either a prostate biopsy or a sample of urine would most accurately determine the level of either 7-hydroxylated steroid or an enzyme capable of catalysing the production of a 7-hydroxylated steroid in the prostate.

It is understood that an abnormal level may be taken to be any level that is either higher or lower as compared to normal levels as determined from a healthy patient. If a difference between the level detected in the patient and the normal level is noted then the patient may either be administered the appropriate treatment for example a 7-hydroxylated steroid or other suitable agent or an enzyme capable of catalysing the production of a 7-hydroxylated steroid or may be referred for further tests.

A normal sequence may be taken to be that which encodes a functional enzyme capable of catalysing the production of a 7-hydroxylated steroid or a sequence that does not comprise a mutation which affects the expression of said functional enzyme. A mutation may be taken to be a deletion, substitution, inversion or translocation.

Examples of methods used to detect a level of a 7-hydroxylated steroid or an enzyme capable of catalysing the production of a 7-hydroxylated steroid would include, capture, direct or indirect enzyme-linked immunosorbent assay (ELISA) wherein, for example, either an antibody specific to the 7-hydroxylated steroid or an antibody reactive to the enzyme capable of catalysing the production of a 7-hydroxylated steroid, for example CYP7B, is bound to a microtitre plate or other suitable item, and the sample to be analysed is applied for an appropriate length of time. An appropriate length of time would be such that an interaction between the antibody and its epitope occurs. After capture of either the 7-hydroxylated steroid or the enzyme capable of catalysing the production of a 7-hydroxylated steroid, a secondary antibody, specific to said steroid or said enzyme, is applied for a suitable length of time. Antibody antigen interactions may then be detected with the use of an antibody capable of interaction with the secondary antibody and conjugated to an enzyme capable of reporting a level via a colourmetric chemiluminescent reaction. Such conjugated enzymes may include but are not limited to Horse Radish Peroxidase (HRP) and Alkaline Phosphatase (AlkP). Other types of conjugated molecule may include fluorescent or radiolabelled antibodies.

Other means of detecting a level of an enzyme capable of catalysing the production a 7-hydroxylated steroid include Western blot and other associated techniques, RT-PCR, PCR, quantitative PCR, quantitative RT-PCR (as defined in Maniatis), Spectrophotometric and Enzymatic reactions well known to those skilled in the art.

Detection of an abnormal sequence may be achieved through techniques well known in the art, including for example agarose gel electrophoresis, PCR and associated techniques, RT-PCR, Southern blotting, Northern blotting, restriction enzyme analysis and DNA sequencing.

In a further aspect of the present invention there is provided a method of detecting a 7-hydroxylated steroid or an enzyme capable of catalysing the production of a 7-hydroxylated steroid in a patient, comprising administering to a patient an amount of either an antibody or a molecule capable of interacting with a 7-hydroxylated steroid or an enzyme capable of catalysing the production of a 7-hydroxylated steroid and detecting any complex comprising said antibody or molecule and said 7-hydroxylated steroid or enzyme capable of catalysing the production of a 7-hydroxylated steroid.

Detection of said complex may involve use of, for example, said antibody or molecule comprising a radiolabel or said antibody or molecule comprising for example, an isotope such as ¹³Carbon. The levels of 7-hydroxylated steroid or enzyme capable of catalysing the production of a 7-hydroxylated steroid in the body, for example the prostate, may be determined by, for example, Magnetic Resonance Imaging (MRI), magnetic resonance spectroscopy, or Computed Axial Tomography (CAT) scanning.

In another embodiment a primary antibody or molecule capable of interacting with a 7-hydroxylated steroid or an enzyme capable of catalysing the production of a 7-hydroxylated steroid may be administered to a patient and detected using a secondary antibody or molecule capable of interacting with said primary antibody or molecule. In this particular embodiment it would be desirable for the secondary antibody or molecule to be either radiolabelled or comprise an isotope such as ¹³Carbon so as to allow detection by MRI or CAT scanning techniques.

Such a method would allow the detection of levels of either 7-hydroxylated steroids or enzymes capable of catalysing the production of a 7-hydroxylated steroid in, for example, the prostate. Results from such a test may indicate that a patient is healthy, suffering from, predisposed to or convalescing from a hormone dependant cancer or other proliferative disorder.

The enzymes described herein may be used in drug evaluation studies. In an embodiment of this aspect of the invention, a cell or cells obtained from either a normal or a diseased tissue may be used as a basis for an assay for agents that modulate the expression of enzymes capable of catalysing the production of a 7-hydroxylate steroid. Advantageously cell lines derived from healthy or diseased tissue may be used. Examples of cell lines appropriate to such an assay would include, for example, normal human prostate cell line PNT2 (ECCAC No: 95012613), human prostate adenocarcinoma cell line PC-3 (ECCAC No: 90112714) or prostate carcinoma cell line LNCap clone FGC (ECCAC No: 89110211). Such an assay may identify agents for example, small organic molecules or antisense oligonucleotides that are capable of modulating the activity or expression of an enzyme capable of catalysing the production of a 7-hydroxylated steroid. Agents identified by said assay could potentially be administered alone or with either an enzyme capable of catalysing the production of a 7-hydroxylated steroid, or a substrate capable of being converted to a 7-hydroxylated steroid by said enzyme in vivo such that the activity or expression of said enzyme in vivo is modulated.

Thus in a further aspect there is provided an assay for identifying agents capable of modulating the activity of an enzyme capable of catalysing the production of a 7-hydroxylated steroid, wherein said assay comprises the steps of:
a) contacting an agent with a prostate cell comprising an enzyme capable of catalysing the production of a 7-hydroxylated steroid, in the presence of a substrate capable of being converted to a 7-hydroxylated steroid by said enzyme; and
b) detecting an amount of substrate converted to a 7-hydroxylated steroid by said enzyme and comparing said level to a normal level.

It is understood that the agent should be contacted to the chosen cell or cell line in the presence of a substrate under conditions that favour the conversion of the substrate to a 7-hydroxylated steroid.

Examples of methods used to detect the amount of substrate converted to 7-hydroylated steroid would include the use of immunological based assays, for example ELISA as previously described, or any other chemiluminescent, fluorescent, or spectrophotometric assay that would appropriately reveal the level of converted substrate or assays such as, for example, thin layer chromatography, high-performance liquid chromatography and gas chromatography mass spectrometry.

Agents that could be identified by such a method include small organic molecules or antisense oligonucleotides.

It is understood that a “normal level” may be determined as the level of an enzyme capable of catalysing the production of a 7-hydroxylated steroid in a healthy, non-diseased tissue or cell/cell line derived therefrom.

In another aspect there is provided use of agents identified by the above method for the treatment and/or prevention of hormone dependant cancers and other proliferative disorders. Such agents may include small organic molecules or antisense oligonucleotides capable of modulating the activity of modulating the activity of an enzyme capable of catalysing the production of a 7-hydroxylated steroid.

The present invention will now be further described by way of example and with reference to the figures, which show:

FIG. 1: The Steroid Pathway of DHEA

3β-HSD, 3β-hydroxysteroid dehydrogenase; 17β-HSD, 17β-hydroxysteroid dehydrogenase; 7HD, 7α-hydroxyDHEA; A/enedione, 5α-Androstenedione; A/enediol, 5α-Androstenediol; A/anediol, 5α-Androstanediol; E₁, Estrone; E₂, 17β-Estradiol and DHT 5α-dihydrotestosterone.

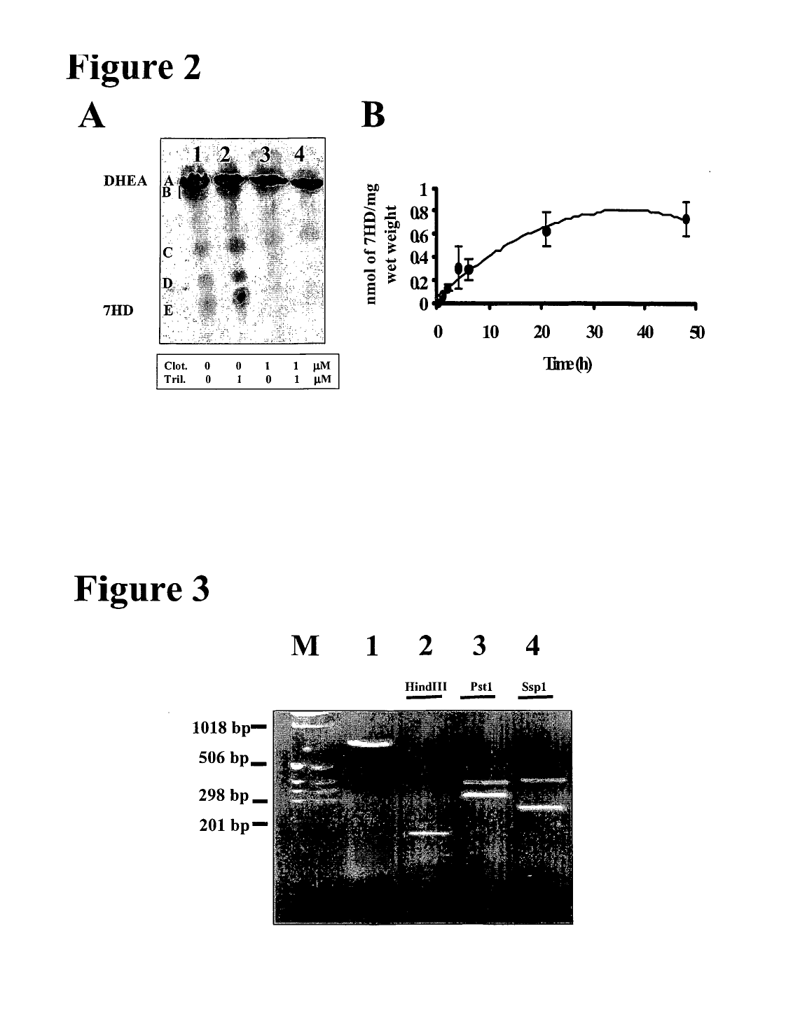

FIG. 2: 7HD is Produced from DHEA in Chips of Human Prostate

(A) TLC analysis of products of DHEA in human prostate chips after 24 h incubation in medium in the absence (lane 1) or in the presence of 1 μM trilostane (lane 2); or 1 μM clotrimazole (lane 3); or 1 μM clotrimazole plus 1 μM trilostane (lane 4).

(B) Time course of production of [¹⁴C]-7HD by human prostate samples (n=2-7).

FIG. 3: RT-PCR Analysis of CYP7B mRNA in Human Prostate

The identity of the PCR product (696 bp) (lane 1) was verified by enzymatic restriction with HindIII (lane 2), PstI (lane 3) and SspI (lane 4), which cut the PCR product at 158 bp, 384 bp and 394 bp, respectively. The nucleic acid size markers are indicated (M).

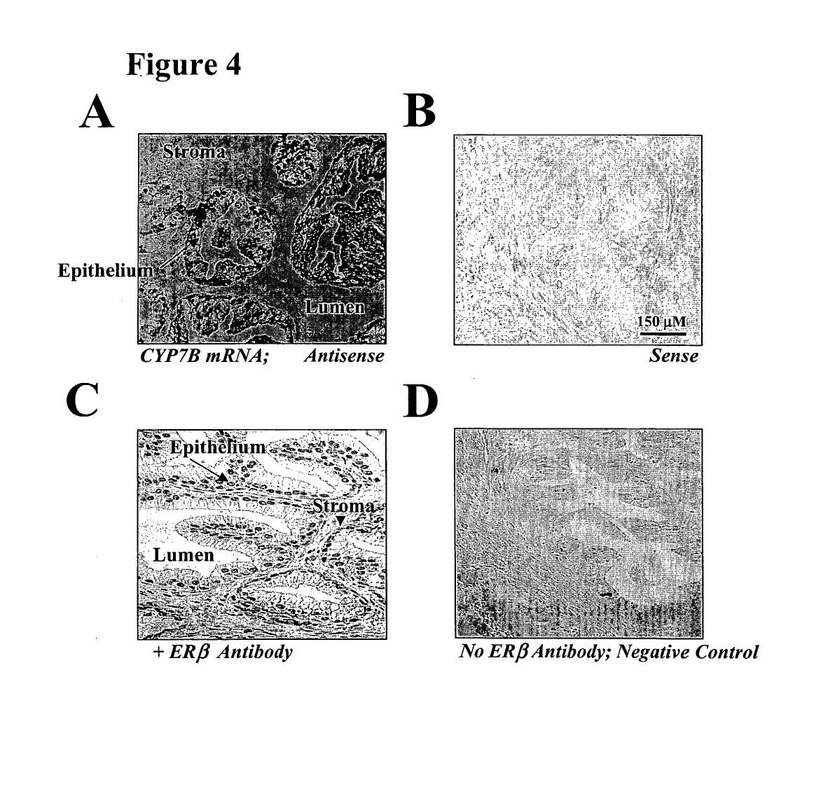

FIG. 4: CYP7B mRNA and ERβ are Co-Localised in Human Prostate Epithelium

Representative high-resolution views of mRNA in-situ hybridisation encoding CYP7B (A) and immunostaining of ERβ (C) in BPH sections. Representative “sense” control sections for CYP7B mRNA and control sections (without primary antibody) for ERβ are shown in (B) and (D), respectively.

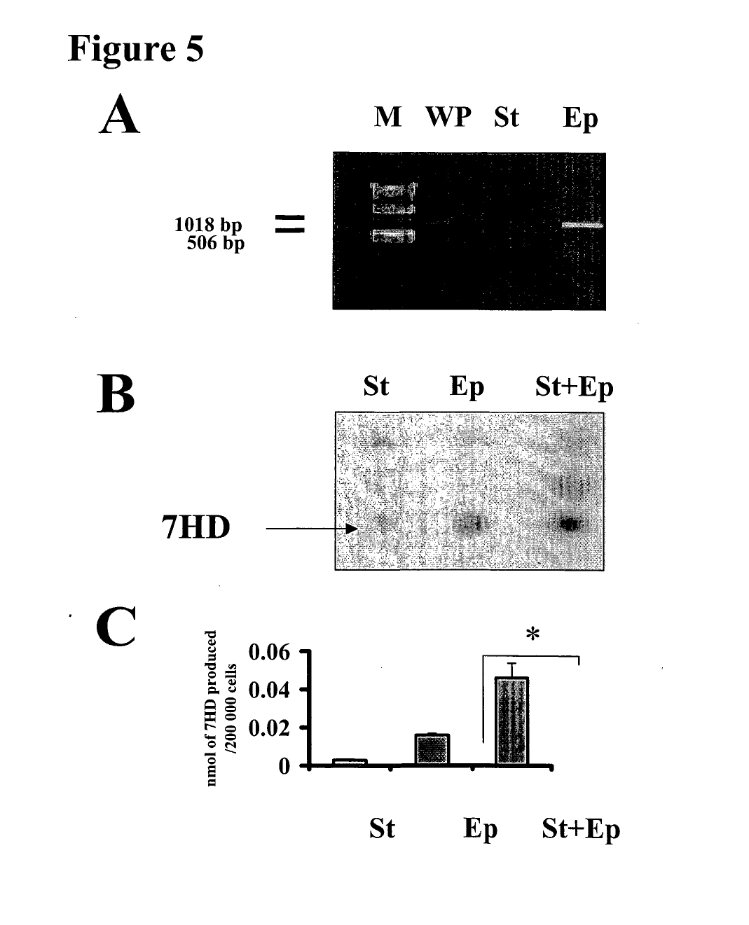

FIG. 5: CYP7B is Expressed in Primary Culture of Epithelial Cells and is Increased by Co-Cultured with Stroma Cells

(A) RT-PCR detection of CYP7B mRNA in whole human prostate (WP, lane 1), primary stromal cells (St, lane 2) and epithelial cells (Ep, lane 3). M, molecular weight markers.

(B) TLC resolution of products generated by 24 h incubation with [¹⁴C]-DHEA of primary culture of stroma cells (St), epithelial cells (Ep) or co-cultured of epithelial and stromal cells (Ep+St). Arrow indicates 7HD.

(C) Production of [¹⁴C]-7HD from DHEA in 24 h by epithelial cells (Ep), stroma cells (St) and co-cultured of epithelial or stromal cells. *, p<0.001, Ep vs St+Ep.

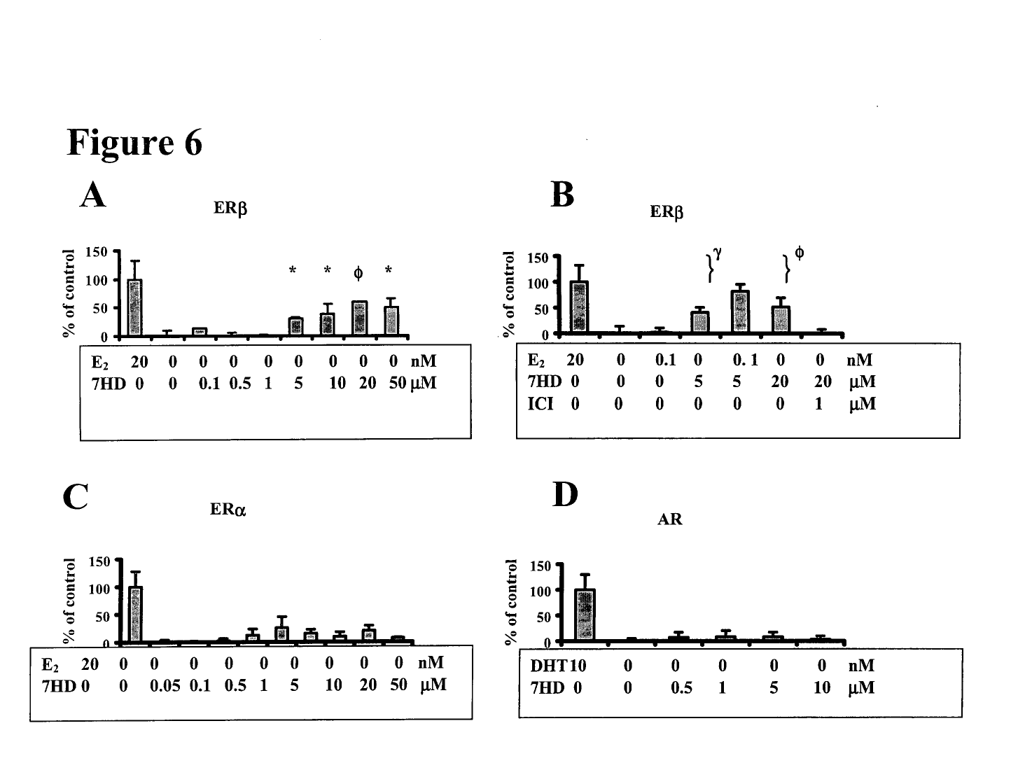

FIG. 6: Transactivation of ERP (A, B), ERa (C) and Androgen Receptor (AR: D), by 7HD.

Values shown are means (±S.E.M.) of 3 to 5 independent experiments each carried out in triplicate. (A) and (B) Transactivation of (ERE)-TK-Luc by ERβ. in HepG2 cells,. C.) Transactivation of (ERE)-TK-Luc by ERα in COS cells and (D) Transactivation of PSA-Luc reporter construct in COS-1 cells containing hAR. Data are presented as the percentage of maximal induction obtained with 20 nM E₂ (hERβ; ×4 fold induction over control;A and B), 10 nM E₂ (mERα; ×7 fold induction over control; C) and 10 nM DHT (hAR; ×17 fold induction over control; D). *, p<0.001; φp<0.05; 7HD vs control without 7HD; [symbol] , p<0.01, E₂ vs 7HD+E₂.

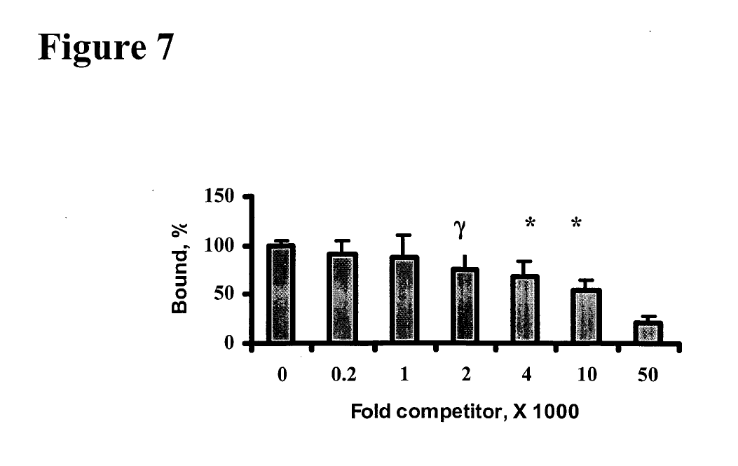

FIG. 7: Competition by 7HD for [3H]-E2 Binding to in vitro Synthesised ERβ Protein.

Reticulocyte lysate containing ERβ protein was equilibrated for 16 h with 5 nM [3H]-E2 and the indicated fold excess of 7HD. Data represent [3H]-E2 bound in presence of 7HD (0-250 μM). [3H]-E2 binding in the absence of 7HD was set at 100%. *, p<0.001 and γ, p<0.01; 7HD vs control without 7HD.

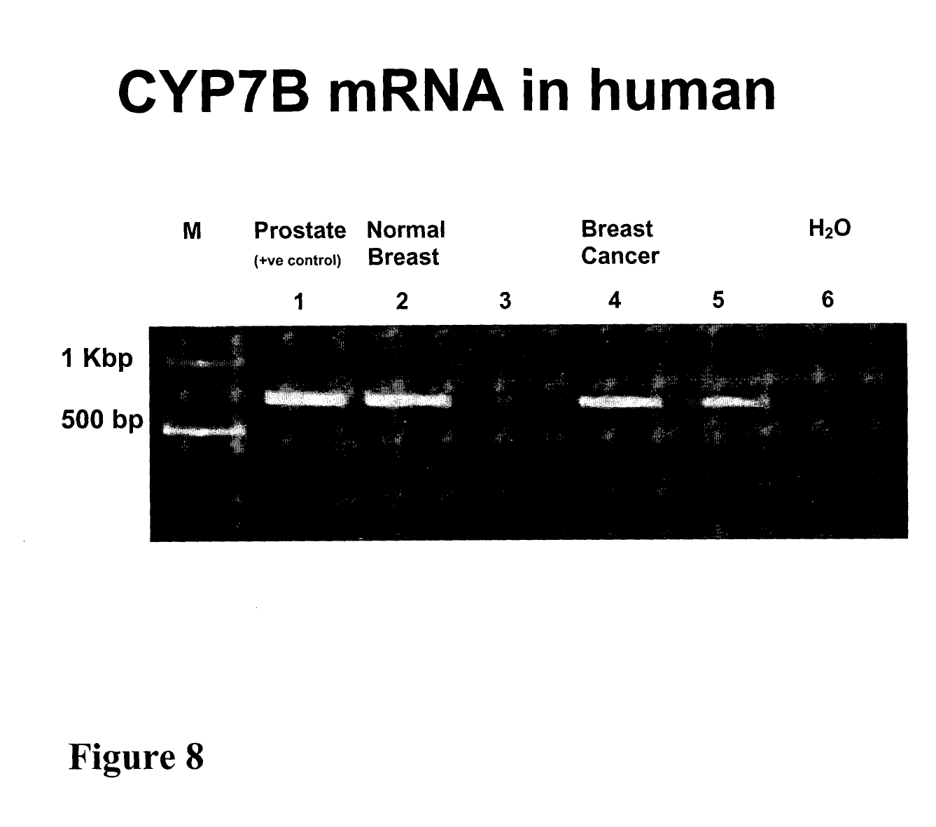

FIG. 8: CYP7B is Expressed in Human Breast

To verify the expression of CYP7B in human breast, RT-PCR was carried out on RNA from four different human breast samples: one sample of normal tissue and 3 samples of breast cancer, either estrogen receptor positive (ER+) or estrogen receptor negative (ER−). Total RNA from human prostate (BPH) was used as a positive control for the PCR and replacement of cDNA by H₂O was used as a negative control to test for contamination. CYP7B-specific primers amplified the expected 696 bp fragment in all samples except H₂O (see FIG. 1). The level of CYP7B mRNA in the human breast samples is high, however one of the breast cancer samples has a much lower level of CYP7B mRNA than the others, suggesting differential expression CYP7B in breast cancers. The implications are that there are variable levels of precursor sex steroid metabolism/activation in breast cancers. This may allow the development of a diagnostic/prognostic test. Lane 2: control normal breast tissue (0.2 μg); lane 3: estrogen receptor positive (ER+) tumour (0.2 μg); lane 4: another ER+ tumour (0.2 μg); lane 5: ER negative tumour (0.2 μg). Lane 1: positive control RNA from human prostate (1 μg); lane 6: negative control water. Note; clear expression of CYP7B mRNA in ER+ and ER− breast cancers.

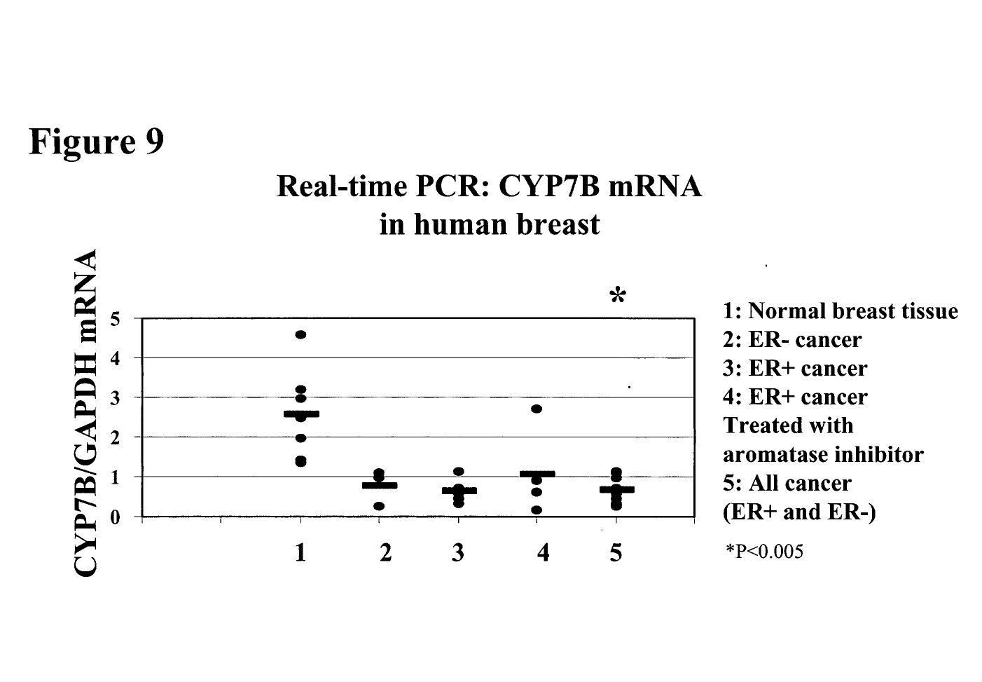

FIG. 9: CYP7B mRNA Expression in Breast Tissue and Breast Cancer

Real-time PCR was used to detect changes in CYP7B mRNA expression in breast cancer. First strand cDNA was produced from 0.2 μg (breast) of total RNA using random primers and Omniscript reverse transcription kit (Qiagen) by standard methods. cDNA reaction (0.2 μl) was then utilised as a template for real-time RT-PCR using Taqman Master Mix and Taqman specific primers for human CYP7B (Hs00191385_ml) as well as Taqman primers for GAPDH as internal standard (all Applied Biosystems). CYP7B mRNA level of expression for each sample was compared with GAPDH mRNA as a housekeeping (invariant) transcript. Data from real-time PCR were analysed and p<0.05 was considered significant. CYP7B mRNA expression was down-regulated in breast cancer for both ER+ and ER− tumours compared with normal breast tissue controls. Treatment of breast cancer with an aromatase inhibitor for two weeks did not alter CYP7B mRNA. The data shows that CYP7B mRNA is expressed in breast cancer, albeit at lower levels than in intact breast. CYP7B mRNA was clearly detected in normal breast tissues and, at a significantly lower level, in breast cancers. Expression of CYP7B mRNA in breast cancer was variable and was not related to estrogen receptor (ER) status. CYP7B mRNA was not altered by aromatase inhibitor treatment for 2 weeks (AR Inh), *P<0.005.

### MATERIALS AND METHODS

Experimental Subjects

Paraffin embedded archival prostate tissues from BPH patients were provided by the Department of Pathology (Western General Hospital, Edinburgh). Fresh prostate samples for CYP7B activity measurements and cell culture were obtained from patients (aged 56-70 years) undergoing transurethral resection of the prostate (TRUP) who have not been treated with hormone ablation. No biopsy samples were used in this study. Randomly selected prostate chips from each specimen were evaluated histopathologically to establish their benign status and the presence of hyperplasia. Only samples taken with informed consent were studied and our protocol was approved by the local Research Ethics Committee.

Steroids.

[1,2,6,7-³H]₄-DHEA (60 Ci/mmol), [4-¹⁴C]-DHEA (53.8 mC/mmol) and [1,2-³H]₂-5α-Androstenediol (A/enediol) (42 Ci/mmol) were purchased from NEN Life Science Products, Boston USA and [2,4,6,7-³H]₄-E₂ was purchased from Perkin Elmer Life Sciences, Boston USA. Non-radioactive steroids and clotrimazole were obtained from Sigma-Aldrich, Poole, U.K. 7HD was purchased from Steraloids Inc, Newport USA. Trilostane was kindly provided by Sanofi Winthrop Development Centre, Newcastle Upon Tyne, U.K. and ICI 182, 780 was purchased from Tocris, Bristol, U.K.

7α-Hydroxylase Activity.

7α-hydroxylase activity was measured in whole human prostate pieces. Surgical BPH samples were incubated at 37° C. for up to 48 h in RPMI 1640 medium supplemented with 5% charcoal stripped serum (DCC-FCS) and the radiolabelled steroid substrates at a concentration of 0.3 μM. Steroids were extracted from the medium with ethyl acetate, dried, and stored at −20° C. until analysis. Recovery was ˜90% (20). The DHEA to 7HD conversion was assessed by TLC, as previously described (20) and quantified using a phosphorimager (FLA-2000, Fujifilm).

High-Performance Liquid Chromatography.

High-pressure liquid chromatography (HPLC) (Waters) with on-line scintillation counting (Berthold) was carried out with a reverse phase C18 column (Luna, Phenomenex) using a mobile phase (H₂O, Methanol and Acetonitrile, 55:25:20, by vol. at 1 ml/min) which gave retention times for 7HD and DHEA at 7 min and 24 min, respectively (20).

RNA Extraction

Total RNA was isolated from human prostate tissue and cells as described previously (25), resuspended in RNase-free H₂O and stored at −70° C. All samples had intact 18S and 28S RNAs, as judged by ethidium bromide staining after agarose gel electrophoresis.

Oligonucleotide Primers, Reverse Transcription and PCR Amplification of CYP7B cDNA.

5′ and 3′ primers for PCR (Oswel DNA Service, Southampton, U.K.) were 5′-dAAGCCTAAATGATGTGCTCC-3′ and 5′-dGAGTGGTCCTGAACTTACG-3′, corresponding to nucleotides 329-347 and 1006-1025 respectively of the human CYP7B cDNA (26). Reverse transcription (RT) was carried out in 20 μl containing 10 mM Tris-HCl (pH 9.0), 50 mM KCl, 2.5 mM MgCl₂, 0.1% (w/v) Triton X-100, 1 mM dNTPs, 10 U of RNasin (Promega, Southampton, U.K.), 1 μg total RNA, 12 U AMV reverse transcriptase (Promega, Southampton, U.K.) and 0.1 nmol 3′ PCR primer. Reactions were incubated for 10 min at room temperature, followed by 30 min at 42° C., then 95° C. for 5 min (to inactivate the reverse transcriptase). Subsequent PCR amplification was carried out by adding 80 μl of buffer containing 50 mM KCl, 10 mM Tris-HCl (pH 9.0), 2.5 mM MgCl₂, 0.2 mM dNTPs, 0.1 nmol of 5′ PCR primer and 2.5 U Taq polymerase (Promega, Southampton, U.K.). Following a “hot start” of 5 min at 94° C., 30 cycles of PCR were carried out: 94° C., 1 min; 56° C., 1 min; 72° C., 1 min followed by 72° C., 10 min. Amplified products were analysed by electrophoresis on 1% (w/v) agarose gels. No products were detected in reactions from which reverse transcriptase had been omitted.

Cloning and Sequencing of RT-PCR Products.

PCR products were subcloned into pGEM-(T) easy (Promega, Southampton, U.K.) and sequenced on both strands.

CYP7B Probe for mRNA in situ Hydridisation.

CYP7B PCR fragment subcloned into pGEM-(T) Easy was used as a template for SP6 or T7 RNA polymerase to generate the “antisense” and “sense” cRNA probes as previously described (27).

CYP7B mRNA in situ Hybridisation.

Paraffin embedded sections (5 μm) were deparaffinised in xylene, soaked in phosphate buffered saline (PBS), and treated with proteinase K treatment in PBS (20 mg/ml) for 10 min at 37° C. Sections were fixed in 4% paraformaldehyde (v/v) for 20 min then treated with 0.25% acetic anhydride (v/v) in 0.1 M triethanolamine for 10 min. Hybridisation with digoxigenin labelled riboprobes (DIG) at 50° C. for 14-16 h in moist chamber, RNase A treatment and washing were as described previously (27). Following hybridisation, DIG-labelled riboprobes were visualised using DIG-alkaline phosphatase conjugated antibody 1:2500 (Boehringer Mannheim) for 30 min at room temperature, washed and developed overnight using a Boehringer Mannheim developing reagent. Non-specific hybridisation was determined by incubation with a DIG-labelled “sense” probe under identical conditions.

ERβ Immunohistochemistry.

Immunohistochemistry study was carried out as previously described (28) using a commercial monoclonal antibody against human ERβ (Serotec, Oxford U.K.).

Primary Cell Cultures of Prostate.

BPH chips were used to establish primary cultures of separated stroma and epithelial cells (29, 30). Co-cultures of epithelial and stroma cells was as described previously (30).

Luciferase Reporter Assays.

COS-1 cells and HepG2 cells were maintained in high glucose Dulbecco's minimum essential medium (DMEM) containing penicillin (25 units/ml), streptomycin (25 μg/ml), and 10% fetal calf serum (v/v). Cells were seeded at a density of 5×10⁵ cells/dish and left to adhere overnight. On the day of the transfection, the medium was replaced with DMEM lacking phenol red supplemented with 10% DCC-FCS (v/v). Transfections were carried out using the calcium phosphate procedure according to standard protocols with 10 μg DNA (1 g expression plasmid, 1-5 μg reporter plasmid, 1 μg pCH110 encoding β-galactosidase used as internal control (Pharmacia) and 3-7 μg pGEM3). Expession plasmid were: the mouse ERα receptor (mERα gift from Prof. M. Parker, London U.K. (31)), human ERβ. receptor (hERβ; from Dr R. White, London U.K. (32)) and human androgen receptor (pSVARₒ; Prof. A. Brinkman, Rotterdam Holand). Mouse ERα receptor shows 88% identity with the human ERβ and both species have the same selectivity for the majority of the steroids. Reporter plasmids were: (ERE)-TK-Luc (gift From Dr V. Giguere, Montreal, Canada) for estrogen responsivity and PSA (PSA61-luc, Prof. J. Trapman, Rotterdam Holland) for androgen responsivity. After 24 h, the medium was changed and the cells were treated with steroid (E₂, DHT, 7HD or an appropriate concentration of ethanol). Following cell lysis, luciferase and β-galactosidase activities were measured as described (33). Data are expressed as relative luciferase/β-galactosidase and are means±S.E.M. from at least three independent experiments.

Ligand-Competition Studies.

Human ERβ clone was synthesised in vitro using the TnT-coupled reticulocyte lysate system following manufacturer instructions (Promega, Southampton, U.K.). Translation reaction mixtures were diluted five times with TEDGMo buffer (40 mM Tris/Hcl, pH 7.4/1 mM EDTA/10% (vol/vol) glycerol/10 mMNa₂MoO₄/10 mM DTT) and 0.1 ml aliquots were incubated for 16 h at 4° C. with 0.5 nM [2,4,6,7-³H]₄-E₂ (specific radioactivity 89 Ci/mmol) in presence of either 0, 1, 5, 10, 20, 50 and 250 μM of 7HD. Bound and unbound steroids were separated by filtration.

Statistics.

Statistical comparisons (‘Sigma Stat’) were by analysis of variance (ANOVA) and the Rank Sum Test. Significance was set at p<0.05.

Results

### EXAMPLE 1

### P450-Dependent Production of 7HD from DHEA by Human Prostate

To determine whether CYP7B activity is present in human prostate, we measured DHEA metabolism in whole prostate chips. DHEA has been reported previously to be the best substrate for recombinant CYP7B in vitro (19). 7α-hydroxylation of DHEA and A/enediol was clearly detectable in chips of whole human prostate and was time-dependent (FIG. 2). The radioactive compound marked “E” (FIG. 2) co-migrated in an identical manner with unlabelled commercial reference compound, 7α-hydroxyDHEA, with the major product of DHEA metabolism by protein extracts from HeLa cells transfected with recombinant CYP7B and also with the rat prostate product of DHEA metabolism, as previously described (20) (data not shown). A/enediol, A/enedione (both in spot “B”) were also produced from DHEA, as previously reported in prostate tissue (34). For a better separation of the different labelled compounds produced during the reaction, we analysed them by HPLC using cold steroid standards. Of all the DHEA metabolites detected by HPLC, more than 50% were a product of CYP7B (9% represent A/enetriol) and only 37% were products of 3β-HSD and 17β-HSD (result not shown). 7α-hydroxylation of DHEA by human prostate chips was inhibited by clotrimazole (1 μM) confirming that the production of 7HD was P450-dependent (FIG. 2). Production of the minor products “C” and “D” was also reduced in presence of clotrimazole, suggesting that they are also the products of a P450 enzyme. Trilostane, which specifically inhibits 3β-HSD activity, increased radioactive 7HD production by 40% (FIG. 2), though it successfully blocked the production of A/anedione. Minor products “C” and “D” were not affected by trilostane. The 7α-hydroxylation of DHEA observed in human prostate was consistent with it being the product of CYP7B metabolism.

### EXAMPLE 2

### CYP7B is Expressed in Human Prostate

To verify the expression of CYP7B in prostate, RT-PCR was carried out on RNA from four different human prostate samples. CYP7B-specific primers amplified the expected 696 bp fragment (FIG. 3). The identity of the PCR product was verified by digestion with HindIII, PstI and SspI, which produced the predicted fragments (FIG. 3). Sequencing of the subcloned PCR product confirmed its identity as human CYP7B (26).

### EXAMPLE 3

### CYP7B mRNA is Co-Localised with ERβ Immunoreactivity in Human Prostate

To determine the site of CYP7B mRNA expression in the human prostate, in situ mRNA hybridisation was carried out on paraffin-embedded sections of prostate using cRNA probe generated from the subcloned PCR product. CYP7B mRNA was highly expressed in the epithelium with very little expression in the stroma and in the vasculature (FIG. 4A). Control sections hybridised to “sense” RNA probe showed low background levels of hybridisation (FIG. 4B). We also determined the localisation of ERβ in human prostate samples using a specific ERβ antibody (FIG. 4C). Interestingly, ERβ was also expressed in the epithelial cells, predominantly in basal regions of the epithelium as confirmed by high molecular weight cytokeratins labelling. This result suggests a co-expression of ERβ with CYP7B.

### EXAMPLE 4

### CYP7B mRNA Expression is Maintained in Primary Epithelial Cells Culture and is Increased by Co-Culture of Stroma with Epithelial Cells

Both CYP7B mRNA and 7α-hydroxylase activity were detected in primary culture of human prostate epithelial cells (FIG. 5). Moreover, epithelial CYP7B activity was enhanced after 5 days of co-culture of epithelial cells with stroma cells (p<0.001; FIG. 5C) suggesting that high epithelial expression of CYP7B is dependent on a diffusible factor produced by co-culture of stroma and epithelial cells. No CYP7B mRNA was found in the stroma cells alone, consistent with the in situ hybridisation findings that CYP7B mRNA is restricted to the epithelium.

### EXAMPLE 5

### 7HD Activates ERβ but Not ERα or AR

To further assess the possible function of the CYP7B product 7HD, we analysed its ability to transactivate ERα, ERβ and AR in a co-transfection assay with estrogen and androgen-responsive reporter genes. Both COS-1 cells and HepG2 cells were used and both these cell lines require exogenous AR, ERα and ERβ to activate androgen (PSA) or estrogen (ERE) responsive reporter genes. In primary experiments, maximal activation of ERα by E₂ was found with 10 nM E₂, whereas maximal activation of ERβ was obtained with 50 nM E₂. Remarkably, 7HD also activated ERβ (FIG. 6A and 6B). 7HD significantly activated ERβ-mediated transcription with an EC₅₀ of 6.2 μM. 7HD transcriptional activity effect was additive to a sub-minimal dose of E₂ (0.1 nM; p<0.01) (FIG. 6B). This effect was specific for ERβ as a similar concentration of 7HD caused only a minimal, non-specific activation of ERα (FIG. 6C). Moreover, 7HD was unable to activate androgen receptor dependent transcription of a PSA-luciferase reporter, whereas 5α-DHT clearly produced strong activation (FIG. 6D). To confirm that 7HD is activating transcription through ER, we used the specific antiestrogen ICI 182, 780. As expected, ICI 182, 780 (1 μM) alone could not stimulate ERα or ERβ activity, but completely abolished both E₂ and 7HD-induced transcription of luciferase in cells co-transfected with ERα or ERβ receptors (FIG. 6B).

The ability of 7HD to inhibit [³H]-E₂ binding was also measured by a competition binding assay using human ERβ receptor synthesised in vitro from our human ERβ clone (hERβ) using a TnT-coupled reticulocyte lysate system (FIG. 7). In our competition study, 7HD interacted with ERβ receptor and inhibited [³H]-E₂ binding dose-dependently.

Discussion

Oxysterol 7α-hydroxylase (CYP7B) is expressed in human prostate, and this pathway is responsible for more than 50% of the DHEA metabolism in this organ. In contrast, metabolism of DHEA towards classical androgens and estrogens forms a relatively minor pathway. Furthermore, it is shown by the present inventors that 7HD is a specific agonist for ERβ but not for ERα or AR, suggesting that 7HD may act as an endogenous ligand for ERβ, in the human prostate. Together the data support the notion that CYP7B generates active steroids within the prostate that may affect the intracrine estrogen:androgen balance and potentially pathogenesis.

7α-hydroxylation of DHEA in humans has been known for many years, initially with the identification of 7HD in urine (35, 36) and subsequently with the detection of 7HD production in skin, brain, mammary tissue, and foetal tissues (37, 38). 7α-hydroxylation of 3β-hydroxysteroids in prostate has been previously described, but the enzyme(s) responsible were hitherto unidentified (24, 39). In the present study, we used a combination of RT-PCR, in situ hybridisation, biochemical and cell culture approaches to show that CYP7B mRNA along with its functional enzyme activity, is localised to the epithelium. Whilst this report confirms that CYP7B is a prime candidate to catalyse the 7α-hydroxylation in prostate, other enzymes might conceivably also be involved. However, mice lacking CYP7B show absolutely no residual 7α- (or 7β-) hydroxylation of DHEA in prostate (22) confirming that CYP7B is the only enzyme involved.

7α-hydroxylation of DHEA is restricted to prostate epithelial cells in vivo and in vitro. This is the first report demonstrating a steroid metabolising enzyme associated exclusively with one type of tissue in the prostate, raising the possibility that 7HD activity might be confined exclusively to the epithelium. Our co-transfection assays show that 7HD is able to activate ERβ which is also localised in the epithelium (12, 13). At sub-minimal concentrations of E₂, 7HD effect on ERβ is additive to E₂. Although 7HD was clearly much less potent than E₂, it achieved similar maximal activation of ERβ. Given that DHEA, and its sulphate, circulate at micromolar concentrations, whereas serum estrogens are at picomolar levels, there is a clear possibility that CYP7B generates sufficient 7HD within the prostatic epithelia to activate ERβ over and above that achieved the very low concentrations of intra-prostatic E₂ (40). Indeed, it is conceivable that 7HD may be a more important ERβ ligand than E₂ itself. A similar proposal has been advanced for A/anediol, which is also an ERβ ligand locally produced in the prostate (23, 41). The data reported herein support the notion that DHEA is a prohormone and 7HD is its active metabolite. In the presence of CYP7B, DHEA is metabolized to an estrogenic steroid acting as an ERβ agonist, 7HD, which may influence the prostatic growth and pathogenesis.

Interestingly, epithelial CYP7B activity was enhanced by co-culture of epithelial and stroma cells. Whether this reflects a differentiation effect in epithelia in co-cultures or is a result of a “crosstalk” signalling between stromal and epithelial cells is uncertain. Previous characterisation of prostate co-cultures suggest the presence of diffusible factors produced by one cell type, which in turn influence the differentiation and gene expression of the other (30, 42, 43). It is conceivable that the “crosstalk” between stromal and epithelial components of the prostate is an important regulator of DHEA metabolism, and may therefore modulate the hormonal status of the gland.

The possibility of a biological role for 7HD, in prostate and elsewhere has so far been unconfirmed and any attempt to elucidate this problem has been hampered by the lack of an established receptor for 7HD. Recent studies have shown that CYP7B expression decreases during development in rodent (44), and is also altered by stress and in Alzhemier's Disease (45, 46), suggesting that the expression of CYP7B may change in response to environmental signals and during aging at least in brain. Loss of prostatic CYP7B may alter the balance between estrogens and androgens, favouring androgenic over estrogenic pathways, by reducing synthesis of the selective ERβ-agonist. Concomitantly any decrease of CYP7B expression increases the availability of native DHEA within the prostate for synthesis of potent androgens. The exact effects of 7HD binding to ERβ on human prostate epithelium and whole prostate are still unknown. One possible role for ERA, as shown in bone, is to modulate ERα-mediated gene transcription (47). Reporter gene assays have demonstrated that ERβ has the capacity to repress the transcriptional activity of ERα (48). By binding to ERβ, 7HD can modulate ERα activity in the stroma compartment and therefore can control the growth of the stroma cells. Also, ERβ is suggested to play a role in the differentiation and proliferation of the prostate cells as well as to modulate both the initial phases of prostate carcinogenesis and androgen-dependent tumor growth (49). Thus, CYP7B may have a significant role in the regulation of the intraprostatic concentration of active steroids and may be a useful tool in the prevention or clinical management of prostate diseases.

In conclusion, it has been shown that CYP7B is highly expressed in both human breast and prostate. Moreover, CYP7B mRNA is differentially expressed in breast and prostate cancer showing that CYP7B may have a significant role in the regulation of the concentration of active steroids within sex steroid sensitive cancer tissues. CYP7B measurement may be of diagnostic or prognostic utility in staging tumours and in guiding therapy.

### REFERENCES

1. Bosland M C 2000 The role of steroid hormones in prostate carcinogenesis. J Natl Cancer Inst Monogr 27: 39-66
2. Schweikert H U, Tunn U W, Habenicht U F, Arnold J, Senge T, Schulze H, Schoder F H, Blom J H M, Ennemoser O, Horniger W, Bartsch G 1993 Effects of estrogen deprivation on human benign prostatic hyperplasia. J Steroid Biochem Mol Biol 44: 573-576
3. Jarred R A, Cancilla B, Prins G S, Thayer K A, Cunha G R, Risbridger G P 2000 Evidence that estrogens directly alter androgen-regulated prostate development. Endocrinology 141: 3471-3477
4. Ferrini R L, Barett-Connor E 1998 Sex hormones and age: a cross-sectional study of testosterone and estradiol and their bioavailable fractions in community-dwelling men. Am. J Epidemiol 147: 750-754
5. Labrie C, Belanger A, Labrie F 1988 Androgenic activity of dehydroepiandrosterone and androsterone in the rat ventral prostate. Endocrinology 123: 1412-1417
6. Nelson L R, Bulun S E 2001 Estrogens production and actions. J Am Acad Dermatol 45: S 16-124
7. Ekman P 2000 The prostate as an endocrine organ: Androgens and estrogens. Prostate 10: 14-18
8. Risbridger G P, Bianco J J, Ellem S J, McPherson S J 2003 International congres on hormonal steroids and hormones ans cancer: Estrogens and prostate cancer. End Related Cancer 10: 187-191
9. Leav I, Ho S M, Ofner P, Merk F B, Kwan P W, Damassa D 1988 Biochemical alterations in sex hormone-induced hyperplasia and dysplasia of the dorsolateral prostates of Noble rats. J Nat Cancer Inst 80: 1045-1053
10. Kuiper G G, Carlsson B, Grandien K, Enmark E, Haggblad J, Nilsson S, Gustafsson J A 1997 Comparison of the ligand binding specificity and transcript tissue distribution of estrogen receptor α and β. Endocrinology 138: 863-870.
11. Brolin J, Skoog L, Ekman P 1992 Immunohistochemistry and biochemistry in detection of androgen, progesterone, and estrogen receptors in benign and malignant human prostatic tissue. Prostate 20: 281-295
12. Horvath L G, Henshall S M, Lee C S, Head D R, Quinn D I, Makela S, Delprado W, Golovsky D, Brenner P C, O'Neill G, Kooner R, Stricker P D, Grygiel J J, Gustafsson J A, Sutherland R L 2001 Frequent loss of estrogen receptor-expression in the prostate cancer. Cancer Res 61: 5331-5335
13. Leav I, Lau K M, Adams J Y, McNeal J E, Taplin M E, Wang J, Singh H, Ho S M 2001 Comparative studies of the estrogen receptors beta and alpha and the androgen receptor in normal human prostate glands, dysplasia, and in primary and metastatic carcinoma. Am J Pathol. 159: 79-92
14. Risbridger G, Wang H, Young P, Kurita T, Wang Y Z, Lubahn D, Gustafsson J A, Cunha G, Wong Y Z 2001 Evidence that epithelial and mesenchymal estrogen receptor-alpha mediates effects of estrogen on prostatic epithelium. Dev Biol 229: 432-442
15. Weihua Z, Makela S, Andersson L C, Salmi S, Saji S, Webster J I, Jensen E V, Nilsson S, Warner M, Gustafsson J A 2001 A role for estrogen receptor beta in the regulation of growth of the ventral prostate. Proc Natl Acad Sci USA 98: 6330-6335
16. Ghanadian R, Puah C M 1981 Relationships between oestradiol-17 beta, testosterone, dihydrotestosterone and 5 alpha-androstane-3 alpha, 27 beta-diol in human benign hypertrophy and carcinoma of the prostate. J Endocrinol 88: 255-262
17. Seckl J R, Walker B R 2001 Steroid metabolism. Bailliere's Clinical Endocrinology and Metabolism. Vol 15.1 Bailliere Tindall, London; 122.
18. Stapleton G, Steel M, Richardson M, Mason J O, Rose K A, Morris R G, Lathe R 1995 A novel cytochrome P450 expressed primarily in the brain. J Biol Chem 270: 29739-45
19. Rose K A, Stapleton G, Dott K, Kieny M P, Best R, Schwarz M, Russell D W, Bjoorkheim I, Seckl J R, Lathe R 1997 Cyp7b, a novel brain cytochrome P450, catalyses the synthesis of neurosteoids 7α-hydroxy-DHEA and 70α-hydroxy-PREG. Proc Natl Acad Sci USA 94: 4925-4930
20. Martin C, Bean R, Rose K, Habib F K, Seckl J R 2001 CYP7B1 catalyses the 7α-hydroxylation of dehydroepiandrosterone and 25-hydroxycholesterol in rat prostate. Biochem J 355: 509-515
21. Li-Hawkins J, Lund E G, Turley S D, Russell D W 2000 Disruption of the oxysterol 7alpha-hydroxylase gene in mice. J Biol Chem 275: 16536-16542
22. Rose K, Allan A, Gauldie S, Stapleton G, Dobbie L, Dott K, Martin C, Wang L, Hedlund E, Gustafsson J A, Seckl J, Lathe R 2001 Targeting the gene encoding mouse neurosteroid hydroxylase CYP7B: vivid reporter activity in dentate gyrus and abolition of a major pathway of steroid and oxysterol hydrolation. J Biol Chem 276: 23937-23944
23. Weihua Z, Lahe R, Warner M, Gustafsson J A 2002 An endocrine pathway in the prostate, ERbeta, AR, 5alpha-androstane-3beta, 17beta-diol, and CYP7B1, regulates prostate growth. Proc Natl Acad Sci USA 99: 13589-13594
24. Li K Foo T, Adams J B 1978 Products of dehydroepiandrosterone metabolism by human mammary tumors and their influence on estradiol receptor binding. Steroids 31: 113-127
25. Chomczynski P, Sacchi N 1987 Single-step method of RNA isolation by acid guanidinium thiocyanate-phenol-chloroform extraction. Anal Biochem 162: 156-159
26. Wu Z, Martin K O, Javitt N B, Chiang J Y L 1999 Structure and functions of human oxysterol 7alpha-hydroxylase cDNAs and gene CYP7B1. J Lipid Res 40: 2195-2203
27. Habib F K, Ross M, Bayne C W, Grigor K, Buck A C, Bollina P, Chapman K 1998 The localisation and expression of 5α-reductase types I and II mRNAs in human hyperplastic prostate and in prostate primary cultures. J Endocrinol 156: 509-517
28. Saunders P T, Millar M R, Williams K, Macpherson S, Harkiss D, Anderson R A, Orr B, Groome N P, Scobie G, Fraser H M 2000 Differential expression of estrogen receptor-α and -β and androgen receptor in the ovaries of marmosets and humans. Biol Reprod 63: 1098-1105
29. Chaproniere D M, McKeelan W L 1986 Serial culture of single adult human prostatic epithelial cells in serum-free medium containing low calcium and a new growth factor from bovine brain. Cancer Res 46: 819-824
30. Habib F K, Ross M, Bayne C W 2000 Development of a new in vitro model for the study of benign prostatic hyperplasia. Prostate 9: 15-20
31. White R, Lees J A, Ham J, Parker M 1987 Structural organization and expression of the mouse estrogen receptor. Mol Endo 1: 735-744
32. Cowley S W, Parker M J 1999 A comparison of transcriptional activation by ER alpha and ER beta. J Steroid Biochem Mol Biol 69: 165-175
33. Leckie C, Chapman K E, Edwards C R, Seckl J R 1995 LLC-PK1 cells model 11 beta-hydroxysteroid dehydrogenase type 2 regulation of glucocorticoid access to renal mineralocorticoid receptors. Endocrinology 136: 5561-5569
34. Mitamura K, Nakagawa T, Shimada K, Namiki M, Koh E, Mizokami A, Honma S 2002 Identification of dehydroepiandrosterone metabolites formed from human prostate homogenate using liquid chromatography-mass spectrometry and gas chromatography-mass spectrometry. J Chromatogr A. 961: 97-105
35. Schneider J J, Lewbart M L 1959 Fractionation and isolation of steroid conjugates. Recent Prog Horm Res 15: 201-230
36. Okada M, Fukushima D K, Gallagher T F 1959 Isolation and characterisation of 3β-hydroxyΔ⁵steroids in adrenal carcinoma. J Biol Chem 234: 1688-1692
37. Farendin I, Fazekas A G, Toth I, Kokai K, Julesz M 1969 Transformation in vitro of [4-¹⁴C]-dehydroepiandrosterone into 7-oxygenated derivates by normal human male and female skin tissue. J Invest Derm 52:357-361
38. Sulcova J, Capkova A, Jirasek J E, Starka L 1968 7-hydroxylation of dehydroepiandrosterone in human foetal liver, adrenals and chorion in vitro. Acta Endocrinol 7: 591-599
39. Sundin M, Warner M, Haaparanta T, Gustafsson J A 1987 Isolation and catalytic activity of cytochrome P-450 from ventral prostate of control rats. J Biol Chem 262: 12293-12297
40. Voigt K D, Bartsch W 1986 Intratissular androgens in benign prostatic hyperplasia and prostatic cancer. J Steroid Biochem 25: 749-757
41. Weihua Z, Makela S, Andersson L C, Salmi S, Saji S, Webster J I, Jensen E V, Nilsson S, Warner M, Gustafsson J A 2001 A role for estrogen receptor β in the regulation of growth of the ventral prostate. Proc Natl Acad Sci USA 98: 6330-6335
42. Bayne W C, Donnelly F, Chapman K, Bollina P, Buck C, Habib F K 1998 A novel co-culture model for benign prostatic hyperplasia expressing both isoforms of 5α-reductase. J Clin Endocrinol Meta 83: 206-213
43. Bayne C W, Ross M, Inglis N F 2003 Induction of 5a-reductse type II mRNA transcription in primary cultured prostate epithelial cells by a soluble factor produced by primary cultured prostate fibroblast cells. Eur J Cancer 39: 1004-1011
44. Bean R, Seckl J R, Lathe R, Martin C 2001 Ontogeny of the neurosteroid enzyme Cyp7b in the mouse. Mol Cell Endocrinol 174: 137-144
45. Graham M, Noble J, Andrew R, Rasmuson S, Olsson T, Secki J, Yau J 2000 Regulation of Cyp7b expression during stress and ageing in rats and Alzheimer's disease in humans. Eur J Neuroscience 12: p184.01 (Abstract)
46. Yau J, Rasmuson S, Andrew R, Graham M, Noble J, Olsson T, Fuchs E, Lathe R, Seckl J R Dehydroepiandrosterone 7-hydroxylase CYP7B: predominant expression in primate hippocampus and reduced expression in Alzheimer's disease. Neuroscience, in press.
47. Lindberg M K, Moverare S, Skrtic S, Gao H, Dahlman-Wright K, Gustafsson J A, Ohlsson C 2003 Estrogen receptor (ER)-β reduces ERα-regulated gene transcription, supporting a “ying yang” relationship between ERα and ERβ in mice. Mol Endocrinol 17:203-208
48. Hall J M, McDonnell D P 1999 The estrogen receptor b-isoform (ERβ) of the human estrogen receptor modulates ERα transcriptional activity and is a key regulator of the cellular response to estrogens and antiestrogens. Endocrinology 140:5566-5578
49. Signoretti S, Loda M 2001 Estrogen receptor beta in prostate cancer: brake pedal or accelerator? Am J Pathol 159:13-1

1-46. (canceled)

47. A method of treating hormone dependant cancers and other proliferative disorders, said method comprising the step of administering to a subject in need thereof, a therapeutically effective amount of an estrogen receptor β (ERβ) modulator.

48. The method of claim 47, wherein the ERβ modulator either antagonizes or agonizes ERβ.

49. The method of claim 47, wherein the ERβ modulator is an ERβ agonist.

50. A method of treating hormone dependant cancers and other proliferative disorders, said method comprising the step of administering to a subject in need thereof, a therapeutically effective amount of 7-hydroxylated steroids and/or enzymes capable of catalyzing the production of a 7-hydroxylated steroid.

51. The method of claim 50, wherein the 7-hydroxylated steroids are 7α-hydroxylated steroids and/or 7β-hydroxylated steroids.

52. The method of claim 50, wherein the 7-hydroxylated steroids are selected from the group consisting of;
i) 7α-hydroxy-DHEA (7DH),
ii) 7α-hydroxy-pregnenolone,
iii) 7α-hydroxy-β-estradiol,
iv) 7α,3β,17β-androstenetriol,
v) 7α,3β,17β-androstanetriol,
vi) 7α-hydroxycholesterol,
vii) 7α-25-hydroxycholesterol,
viii) 7α-24-hydroxycholesterol,
ix) 7α-27-hydroxycholesterol and
x) other 7α-di-hydroxy and 7α-multi-hydroxylated forms of cholesterol.

53. The method of claim 50, wherein the 7-hydroxylated steroids and/or enzymes capable of catalysing the production of a 7-hydroxylated steroid are administered in association with a pharmaceutically acceptable carrier or diluent.

54. The method of claim 50, wherein the 7-hydroxylated steroids and/or enzymes capable of catalysing the production of a 7-hydroxylated steroid are directly or locally administered to the prostate and/or breast.

55. The method of claim 50, wherein the hormone dependant cancer is prostate cancer or breast cancer.

56. The method of claim 50, wherein the proliferative disorder is a disorder of the prostate or breast.

57. The method of claim 56, wherein the disorder of the prostate is a disorder of prostate development or prostate ageing.

58. The method of claim 56, wherein the disorder of the prostate is benign prostatic hyperplasia (BHP) and/or prostatitis.

59. The method of claim 50, wherein the enzyme that produces 7-hydroxylated steroids is oxysterol 7α-hydroxylase (CYP7B).

60. The method of claim 50, wherein the enzyme capable of catalyzing the production of a 7-hydroxylated steroid is modified.

61. The method of claim 60, wherein the enzyme capable of catalyzing the production of a 7-hydroxylated steroid is modified to improve substrate affinity.

62. The method of claim 50, wherein the enzyme capable of catalyzing the production of a 7-hydroxylated steroid is recombinantly or synthetically produced.

63. The method of claim 50, wherein the 7-hydroxylated steroid is provided by contacting an enzyme capable of catalyzing the production of said 7-hydroxylated steroid or a cell comprising an enzyme capable of catalyzing the production of said 7-hydroxylated steroid, with a suitable substrate.

64. The method of claim 63, wherein the cell is transformed with a vector containing a gene encoding an enzyme capable of catalyzing the production of said 7-hydroxylated steroid.

65. The method of claim 63, wherein the suitable substrate is selected from the group consisting of;
i) dehydroepiandosterone (DHEA),
ii) 3β-androstanediol,
iii) 3β-androstenediol; and
iv) β-estradiol

66. The method of claim 50, wherein the enzyme capable of catalyzing the production of a 7-hydroxylated steroid is provided by means of a nucleic acid encoding said enzyme.

67. The method of claim 66, wherein the nucleic acid is contained within a nucleic acid vector.

68. The method of claim 66, wherein the nucleic acid encodes oxysterol 7α-hydroxylase (CYP7B).

69. A method of detecting either a level of a 7-hydroxylated steroid or a level of an enzyme capable of catalyzing the production of a 7-hydroxylated steroid or detecting a mutation in a sequence encoding an enzyme capable of catalyzing the production of a 7-hydroxylated steroid, wherein the method comprises the steps of;
a) providing a sample from a patient;
b) detecting a level of 7-hydroxylated steroid or an enzyme capable of catalyzing the production of a 7-hydroxylated steroid or ascertaining the sequence of the nucleic acid encoding said enzyme; and
c) comparing said detected level or the sequence of said nucleic acid with a normal level or sequence.

70. The method according to claim 69 for use in detecting the efficacy of a drug used to treat hormone dependant cancers and other proliferative disorders.

71. The method of claim 69 for use in ascertaining the stage of a tumor.

72. The method of claim 69, wherein the patient is either a healthy person, a person suspected of having, predisposed to developing, or suffering from a hormone dependant cancers or other proliferative disorder.

73. The method of claim 69, wherein the sample is a biopsy or a body fluid.

74. The method of claim 73, wherein the biopsy is a prostate biopsy or breast tissue biopsy.

75. The method of claim 73, wherein the body fluid is selected from the group consisting of
i) blood;
ii) urine; and/or
iii) semen.

76. The method of claim 69, wherein the normal sequence encodes a functional enzyme capable of catalyzing the production of a 7-hydroxylated steroid.

77. The method of claim 69, wherein the normal sequence is a sequence that does not comprise a mutation which effects the expression of said functional enzyme.

78. The method of claim 69, wherein the level of 7-hydroxylated steroid or an enzyme capable of catalyzing the production of a 7-hydroxylated steroid is detected by means of immunological detection techniques.

79. The method of claim 78, wherein the level of 7-hydroxylated steroid or an enzyme capable of catalyzing the production of a 7-hydroxylated steroid is detected by ELISA or Western blot.

80. The method of claim 69, wherein the level of an enzyme capable of catalyzing the production of a 7-hydroxylated steroid is detected by PCR and associated techniques, for example RT-PCR, quantitative PCR and quantitative RT-PCR.

81. The method of claim 69, wherein the level of an enzyme capable of catalyzing the production of a 7-hydroxylated steroid is detected by spectrophotometric and enzymatic reactions

82. A method of detecting a 7-hydroxylated steroid or an enzyme capable of catalyzing the production of a 7-hydroxylated steroid in a patient, comprising administering to a patient an amount of a molecule capable of interacting with a 7-hydroxylated steroid or an enzyme capable of catalyzing the production of a 7-hydroxylated steroid and detecting any complex comprising said molecule and said 7-hydroxylated steroid or enzyme capable of catalyzing the production of a 7-hydroxylated steroid.

83. The method of claim 82, wherein the molecule capable of interacting with a 7-hydroxylated steroid or an enzyme capable of catalyzing the production of a 7-hydroxylated steroid is an antibody.

84. The method of claim 82, wherein the molecule or antibody further comprises a radiolabel or a radioactive isotope.

85. A method for identifying agents capable of modulating the activity of an enzyme capable of catalyzing the production of a 7-hydroxylated steroid, wherein said assay comprises the steps of:
a) contacting an agent with a prostate cell comprising an enzyme capable of catalyzing the production of a 7-hydroxylated steroid, in the presence of a substrate capable of being converted to a 7-hydroxylated steroid by said enzyme; and
b) detecting an amount of substrate converted to a 7-hydroxylated steroid by said enzyme and comparing said level to a normal level.

86. Use of agents identified by the method of claim 85 for the treatment and/or prevention of hormone dependant cancers and other proliferative disorders.
